(19) United States
(12) Patent Application Publication
(10) Pub. No.: US 2012/0116537 A1
(43) Pub. Date: May 10, 2012

(54) OUTER EAR MUSCULATURE DETECTION MEANS

(76) Inventor: David Liebetanz, Goettingen (DE)

(21) Appl. No.: 13/322,223

(22) PCT Filed: Jun. 3, 2010
(86) PCT No.: PCT/EP2010/003367

§ 371 Date: Dec. 29, 2011

(30) Foreign Application Priority Data
Jun. 15, 2009 (DE) 10 2009 025 313.0

(51) Int. Cl. A61F 2/72; A61F 11/04; A61N 1/36; G05B 15/02

(52) U.S. Cl. 623/25; 700/17; 607/57

(57) ABSTRACT

The invention relates to an auxiliary device for a living being having a) a means for detecting outer ear musculature (3) detecting an activation of at least a part of the outer ear musculature (21 to 28) of a living being, and b) a control device (4) connected to the outer ear musculature detecting means, c) wherein the control device (4) d) is set up for processing the signals detected by the outer ear musculature detecting means (3), and c2) can be controlled for outputting control signals to a technical auxiliary device (5) by means of which the technical auxiliary device (5) can be controlled. A technical area of application of the invention is, for example, neuroprosthetics. Improved opportunities are sought here, in order to derive signals of the nervous system that can be arbitrarily generated or modified. By means of such derived signals, for example, technical auxiliary systems for failed bodily functions (prosthetics) can be controlled.

The invention relates to the detection of an activation of at least part of the outer ear musculature of a living being.

A technical field of application of the invention for example lies in neuroprosthetics. Here, improved options are sought for deriving signals from the nervous system, which signals can be voluntarily generated or modified. By way of example, such derived signals are to be used to control technical aid systems for failed bodily functions (prostheses).

By way of example, DE 101 24 839 A1 has disclosed such a prosthesis control. According to this, a technical option for controlling prostheses consists in deriving muscle signals from the remaining musculature of amputated extremities. The signals can be detected as EMG signals or, as proposed in DE 101 24 839 A1, can be detected by means of optical sensing.

By way of example, the muscle signals can be derived from the shoulder musculature. The technique of targeted muscle reinnervation allows the nerve stumps of the amputated extremity to activate, i.e. stimulate, the proximal musculature. The EMG signals obtained from this correspond to the activation of the amputated extremity. This enables prosthesis control on the basis of the original nerve impulses for the amputated extremity. However, a disadvantage of this technique is that it requires a relatively comprehensive invasive intervention with the corresponding risks. Moreover, the application is limited to amputated extremities. This technique cannot be applied in the case of paralyzed extremities.

The invention is therefore based on the object of disclosing options for controlling aids and other devices, which can be used on humans in a simpler and less invasive fashion.

This object is achieved by an auxiliary device for a living being, with an outer ear musculature detection means which detects an activation of at least part of the outer ear musculature of a living being. The auxiliary device has a control device connected to the outer ear musculature detection means. The control device is embodied to process the signals detected by the outer ear musculature detection means and to output control signals to a technical aid. The technical aid can be controlled by the control signals. In principle, this allows any technical aid to be controlled by the auxiliary device according to the invention. In this context, muscle activation should be understood to mean any muscular stimulation in the broadest sense, particularly also minor muscle stimulations that do not necessarily lead to muscle contractions that are visible from the outside.

The human ear is subdivided into the outer ear, the middle ear and the inner ear. It is well known that the ossicles—malleus, incus and stapes—are situated in the middle ear of a human. Moreover, there are muscles in this region of the middle ear, such as the tensor tympani muscle and the stapedius muscle, which involuntarily contract reflexively if a high noise level is perceived. This reflexive muscle contraction protects the hearing from damage by excessively high noise levels. By way of example, the reflexive contraction of the stapedius muscle, also referred to as the stapedius reflex, is used in the field of cochlear implants for artificially restoring the power of hearing, as described in e.g. DE 10 2007 008 154 A1.

The aforementioned reflex-like muscular reactions in the region of the middle and inner ear cannot be influenced voluntarily by humans and are therefore not suitable for deriving signals, for example for controlling a prosthesis. The present invention is based on the fact that humans also have muscles available in the region of the outer ear—referred to as outer ear musculature below—but no attention has been given to these until now because they are not used under normal circumstances. Humans have nine outer ear muscles on each ear. It was found that, given appropriate training, these muscles can be activated voluntarily by humans.

In contrast to the remaining musculature of the facial musculature innervated by the facial nerve, the outer ear musculature does not have an essential function in humans. Therefore the outer ear musculature is normally only subjected to voluntary movements in a very restricted sense. However, this is cross-striated musculature, innervated by the facial nerve. Accordingly, the present invention proposes technical options for deriving and using signals from the outer ear musculature, for example for controlling prostheses or other technical equipment, both for disabled and for non-disabled persons.

Using the outer ear musculature is advantageous in that the muscles are substantially accessible from the outside and sensor means, e.g. electrodes, can be attached by non-invasive or at least minimally invasive means; hence this is low risk. By way of example, it is feasible that an outer ear musculature detection means with electrodes for detecting the muscle stimulation can be attached in a retro-auricular fashion, i.e. behind the auricle, on the ear of a human using an attachment clip such that the outer ear musculature detection means can be removed at any time. Hence the outer ear musculature detection means according to the invention is very user friendly and can moreover be produced in a simple and cost-effective fashion.

Compared to techniques developed in the field of brain/computer interface (BCI) technology, the invention is advantageous in that it is less error-prone during signal detection. BCI technology is relatively error-prone during signal detection as a result of using a relatively large number of surface electrodes which moreover can only be attached to the body in an unstable fashion. Moreover, this only allows a relatively low data transfer rate. It was also found that patients find it difficult to master voluntary influencing of brainwaves. A further disadvantage lies in the fact that patients cannot carry out other activities in parallel while using a BCI, because this influences the brainwaves in a different fashion and reduces the signal quality.

In contrast thereto, the invention offers the advantage of being able to use body elements of the living being which, on the one hand, are relatively accessible and can be trained in respect of voluntary stimulation and, on the other hand, are not required for other elements of the daily routine. As a result of the relatively high number of muscles available in the outer ear, namely nine per ear, there can at most be an 18-channel control of aids without having to restrict other activities in the process.

A number of specific methods were developed for training the muscles in the outer ear; these methods make the outer ear musculature sufficiently accessible to voluntary movement so that a number of the outer ear muscles can be activated voluntarily and independently of one another. Only this provides a comparatively high number of control channels (1 to at most 9 per side) for the system. The methods serve for voluntary recruitment of the outer ear musculature by neuroplastic reorganization of the motor-cortical representation. The methods developed for this can be used as a whole sequence, in combination or else on their own.
A) The cortical representation of the musculature innervated by the facial nerve, including the outer ear musculature, is charted with the aid of transcranial magnetic stimulation (TMS) and/or by means of functional magnetic resonance imaging.
B) Paired associative stimulation and transcranial direct current stimulation is used for a focused increase in the cortical excitability before and during the training phase (explained below in D) and E)). After the cortical representation area of the individual outer ear muscles has been charted in the individual patient in step A), a series of TMS pulses are applied over the respective area, with an electric pulse via the facial nerve in each case preceding these by a certain amount of time. This brings about a targeted increase in the excitability of the cortical neurons that activate the outer ear muscles. As a result of the lowering of the excitation threshold caused by this, these muscles can now be voluntarily activated in a simpler and more targeted fashion.
C) After an upper extremity has been amputated, the facial representation extends into the former area of the hand. This may lead to misperceptions in the phantom limb by the face being touched (Birbaumer et al., 1997). Animal experiments were also able to demonstrate that nerve lesions induce a rapid expansion of intact motor representations in cortical areas, which are disconnected from the periphery (Sanes et al., 1988; Donoghue et al., 1990). Removing the local inhibition and changing the synaptic effectiveness reactivates latent corticocortical connections in the process (Jacobs and Donoghue, 1991). Interestingly, the disconnected cortex exhibits increased modifiability by otherwise subliminal stimulation (Ziemann et al., 1998).
On the basis of these neuro-scientific principles, a further method achieves a transient surrounding denervation in parts of the facial musculature by means of local injections of botulinum toxin, which reaches the outer ear muscles and brings about a reduction in the synaptic effectiveness of the injected musculature. This results in an expansion and unmasking of the cortical ear muscle representation. This effect lasts for a few months. The remaining methods (A, B, D and E) should be applied during this time in order to achieve an enduring voluntary control of the outer ear musculature.
D) The most important method of targeted voluntary recruitment is the training under EMG feedback with visual and/or acoustic feedback, for example supported by training software running on a computer.
E) The effectiveness of training under EMG feedback can be further increased by mechano-sensory feedback. Here, the individual outer ear muscle to be trained is, analogously to the visual or acoustic feedback, stimulated mechanically during the muscle activation; this stimulation is external and brought about by means of a vibrator or an electrical stimulus with a frequency of between 2 and 50 Hz. As a result there is direct afferent feedback of the muscular activity via mechano-receptors of the skin and musculature and there is a stabilization of the reduced activation threshold.

A further advantage of the invention consists of the fact that using the EMG signals from the outer ear musculature for controlling prostheses or technical aids for rehabilitation allows for care of patients with very different paralysis and amputation patterns right up to severe tetraplegia. The fact that even paraplegics are generally able to activate the outer ear musculature is advantageously utilized. A further significant advantage lies in the fact that the system can be used independently of other activities (e.g. also while running or talking). Moreover, implanting epimysial EMG electrodes or an amplifier/transmitter unit only requires a minimally invasive, low-risk intervention in contrast to e.g. implanting subdural electrodes or to relatively serious interventions within the scope of targeted muscle reinnervation.

In detail, the outer ear musculature detection means can have varying designs. What is decisive is that an activation of at least part of the outer ear musculature of a living being, i.e. of a human or an animal, is detected.

According to an advantageous embodiment, the outer ear musculature detection means has a sensor means for detecting the electric muscle activity of at least one outer ear muscle. Detecting an electric signal on the body of a human is advantageous in that use can be made of electrodes with relatively simple designs and that no other complicated apparatuses are required. Moreover, this advantageously immediately provides electric signals that can be processed further and amplified electrically. Advantageously, e.g. EMG electrodes can be used as sensor means. EMG is an abbreviation for electromyography, which, in human medicine, is an electrophysiological method of diagnostics in neurology, in which electric muscle activity is measured. By way of example, potential variations of individual muscles can be derived with the aid of needle electrodes. Special needles even allow the detection of individual muscle fibers. However, the sensor means also comprise EMG electrodes embodied as surface electrodes, which generate signals by measuring changes in the potential on the skin. Such EMG signals detect the electric activity of the muscle depending on the strength of the muscle contraction. Hence graded signals can be derived depending on the strength of the muscle contraction. Endomysial or epimysial electrodes, which are accordingly implanted under the skin, can be used for chronic derivation of EMG signals.

In a further embodiment, the outer ear musculature detection means has a sensor means for detecting a mechanical movement triggered by an ear muscle. Suitable sensor means for this include e.g. force sensors, such as piezosensors or strain gauges, or optical sensors, as disclosed in DE 101 24 839 A1. To this end, a reference pattern can be arranged on the skin surface in the region of the muscle to be detected and the deformation of said reference pattern is detected by the optical sensor. By way of example, the optical sensor can be a CCD camera sensor or a photocell matrix.

In a further advantageous embodiment, the outer ear musculature detection means has at least one light source and a light detection means. Here, the light source can be arranged on one side of the auricle and the light detection means can be arranged on the other side of the auricle, such that a light beam is routed through the tissue of the auricle. Muscle contractions can likewise be detected as a result of such transillumination. The muscle density in a contracted muscle is increased compared to a non-contracted muscle, and so the light intensity arriving at the non light detection means decreases.

The aforementioned embodiments of the sensor means and optionally further embodiments can also be used in combination in the outer ear musculature detection means according to the invention, with the best-suited sensor principle being selected for the respective muscle to be detected.

According to an advantageous development of the invention, the outer ear musculature detection means has a first interface device for wireless data transmission for outputting detected muscle signals to the control device. By way of example, the interface device may be embodied as a Bluetooth interface or a comparable radio transmitter. The interface device can be used to transmit the detected signals from the outer ear musculature to the control device, which processes the signals and, for example, controls a prosthesis. The wireless interface allows a compact design of the outer ear musculature detection means and makes it possible to dispense with a cabled connection, considered bothersome, between the outer ear musculature detection means and the control device.

Advantageously, the outer ear musculature detection means can be designed as a pure detection means, i.e. as a sensor system, and contain no further components, for example for a prosthesis control. An integrated design of the outer ear musculature detection means and the control device as a unit is also advantageous. In an advantageous embodiment of the invention, the outer ear musculature detection means has an integral design with another apparatus, which is embodied to perform other functions as well. An apparatus to be worn on the ear of a living being, with at least one outer ear musculature detection means according to the above-described principle and a further functional means for carrying out another function not directed to detecting the ear musculature, is advantageous. By way of example, the further functional means can be a hearing aid. Thus the outer ear musculature detection means can be integrated into a hearing aid to be worn on the ear. Integrating or attaching the outer ear musculature detection means to a spectacle frame, a Bluetooth hands-free device to be worn on the ear or a headset is also advantageous.

An advantageous auxiliary device for a living being has an outer ear musculature detection means according to the above-described principle and a control device connected thereto. The control device is designed to process the signals detected by the outer ear musculature detection means and to output control signals to an aid. The auxiliary device can be embodied as a compact apparatus to be attached in the region of the ear of a living being, with an integral design of the outer ear musculature detection means and the control device. The control device can also be arranged separately on the body of the living being. The aid can advantageously be a support means for a disabled person, for example a prosthesis, a wheelchair or a voice computer. Advantageously, the aid can also be any other piece of equipment from everyday life, e.g. a telephone, a headset or a computer game.

The muscle signals detected by the outer ear musculature detection means can advantageously also be used to control domestic appliances, lifts or other equipment to be operated by humans, such as e.g. computers or musical instruments. It is possible to see that the invention is fundamental in nature and permits a very broad scope of application. The invention can advantageously support both disabled and non-disabled persons.

According to an advantageous development of the invention, the auxiliary device has a second interface device for wireless data transmission for outputting the control signals to the aid. The wireless second interface device can for example be embodied as a radio interface, e.g. Bluetooth. This in turn reduces the required circuitry complexity.

The aid that is controllable by the control signals advantageously is an aid that is controllable by electric signals.

According to an advantageous development of the invention, the outer ear musculature detection means is designed such that it can be partly or wholly implanted into the living being. In the case of being partly implantable, it is advantageous to design at least the electrodes for detecting the activation of the ear musculature in an implantable fashion. According to an advantageous development of the invention, the whole auxiliary device, including the control device, is embodied as a system that can be implanted into the living being.

According to an advantageous development of the invention, the auxiliary device is embodied to output control signals to a cochlear implant. Here, the auxiliary aid has a design that is compatible with the cochlear implant in respect of the control signals such that a cochlear implant, which receives the control signals from the auxiliary device, can be controlled. By way of example, the control signals can cause the cochlear implant to switch between different operating modes, e.g. from a normal mode to a telephone mode, which makes it easier for a user of the cochlear implant to telephone effectively, even when there is interfering noise from the surroundings. In particular, the reception frequency band of the cochlear implant can in the process be matched to the voice band transmitted by a telephone, such that surrounding noises outside of the voice band are suppressed.

According to an advantageous development of the invention, the auxiliary device is embodied to output control signals to an arm prosthesis. This allows a living being using the auxiliary device to control the arm prosthesis by activating the outer ear musculature.

In the following text and using a drawing, the invention will be explained in more detail on the basis of an exemplary embodiment.

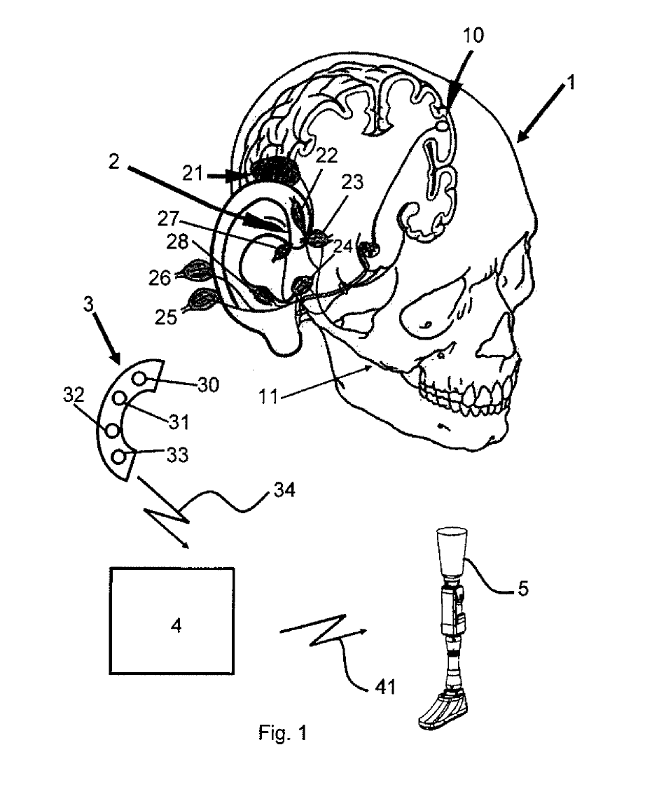

FIG. 1 shows an auxiliary device for a human with an outer ear musculature detection means arranged on the outer ear.

FIG. 1 shows the head 1 of a human, an ear 2, an outer ear musculature detection means 3, a control device 4 and a leg prosthesis 5 as an aid. The motor-cortical representation area 10 and the facial nerve 11 are also illustrated in respect of the head 1. In respect of the outer ear 2, muscles 21, 22, 23, 24, 25, 26, 27, 28 are illustrated in an exemplary fashion as parts of the outer ear musculature of a human.

The outer ear musculature detection means 3 is provided for detecting an activation of at least parts of the outer ear musculature. By way of example, the outer ear musculature detection means 3 is embodied in the form of a clip to be worn on the outer ear, which clip e.g. has a shape comparable to a hearing aid. The outer ear musculature detection equipment has sensor means 30, 31, 32, 33, which are used in this embodiment to detect four of the aforementioned muscles 21, 22, 23, 24, 25, 26, 27, 28. The outer ear musculature detection means 3 furthermore has a battery for power supply, a multichannel EMG amplifier for amplifying the signals from the sensor means 30, 31, 32, 33, optionally analog/digital converters for digitizing the signals and a microprocessor for processing the signals. Moreover, provision is made for an interface device for wireless data transmission, which is used to transmit data to the control device 4 via a data transmission path 34.

The control device 4 processes the data received from the outer ear detection means 3 and uses this to determine control signals for controlling the leg prosthesis 5. In the exemplary embodiment, the control signals are transmitted to the leg prosthesis 5 via a further wireless interface device via a data transmission path 41.

By voluntarily activating specific parts of the outer ear musculature, the user can now control the leg prosthesis 5 as desired.

1. An auxiliary device for a living being, with
a) an outer ear musculature detection means (3) which detects an activation of at least part of the outer ear musculature (21 to 28) of a living being and
b) a control device (4) connected to the outer ear musculature detection means,
c) wherein the control device (4) is embodied
c1) to process the signals detected by the outer ear musculature detection means (3) and
c2) to output control signals to a technical aid (5), by means of which the technical aid (5) can be controlled.

2. The auxiliary device as claimed in claim 1, wherein the outer ear musculature detection means (3) has a sensor means (30 to 33) for detecting the electric muscle activity of at least one ear muscle (21 to 28).

3. The auxiliary device as claimed in claim 1, wherein the outer ear musculature detection means (3) has a sensor means (30 to 33) for detecting a mechanical movement triggered by an ear muscle (21 to 28).

4. The auxiliary device as claimed in claim 1, wherein the outer ear musculature detection means (3) has at least one light source and a light detection means.

5. The auxiliary device as claimed in claim 1, wherein the outer ear musculature detection means (3) has a first interface device for wireless data transmission for outputting detected muscle signals to the control device (4).

6. The auxiliary device as claimed in claim 1, wherein the aid (5) is a support means for a disabled person, more particularly a prosthesis, a wheelchair or a voice computer.

7. The auxiliary device as claimed in claim 6, wherein the auxiliary device has a second interface device for wireless data transmission for the outputting control signals to the aid (5).

8. The auxiliary device as claimed in claim 1, wherein the outer ear musculature detection means is designed such that it can be partly or wholly implanted into a living being.

9. The auxiliary device as claimed in claim 1, wherein the auxiliary device is embodied to output control signals to a cochlear implant, with the control signals being control signals that are compatible with the cochlear implant.

10. The auxiliary device as claimed in claim 1, wherein the auxiliary device is embodied to output control signals to an arm prosthesis, with the control signals being compatible with the arm prosthesis.
